(19) United States
(12) Patent Application Publication
(10) Pub. No.: US 2015/0217064 A1
(43) Pub. Date: Aug. 6, 2015

(54) Aromatherapy Vaporization Device and Method

(71) Applicant: MICHAEL ALEXANDER TRZECIESKI, MID LEVELS (HK)

(72) Inventor: MICHAEL ALEXANDER TRZECIESKI, MID LEVELS (HK)

(21) Appl. No.: 14/614,005

(22) Filed: Feb. 4, 2015

Related U.S. Application Data
(60) Provisional application No. 61/935,349, filed on Feb. 4, 2014.

(51) Int. Cl. A61M 15/00; A61M 16/14; A61M 16/10

(52) U.S. Cl. CPC A61M 15/0041; A61M 15/0035; A61M 16/109; A61M 16/14; A61M 15/0086; A61M 2202/0468; A61M 2205/3673; A61M 2205/3368; A61M 2205/8206

(57) ABSTRACT

A novel aromatherapy vaporization device having a vaporization chamber and an inhalation aperture is disclosed. Phyto material is disposed in an aromatherapy material container, which is inserted into the vaporization chamber and a spike is heated to a predetermined temperature. The spike is then inserted into the aromatherapy material container and the spike contacts the phyto material and causes phyto material vapor to be emitted into the vaporization chamber which is then available for inhalation from the inhalation aperture.

### CROSS REFERENCE TO RELATED APPLICATION

This application benefits from the priority of U.S. Provisional Applications 61/935,349 filed on Feb. 4, 2014, which is incorporated herein by reference.

### TECHNICAL FIELD OF THE INVENTION

The technical field relates to a device and method that provides for vaporization of phyto materials.

### BACKGROUND OF THE INVENTION

Aromatherapy generally uses essential oils, which are extracted from phyto materials, such as leaves of plants, for therapeutic benefits. These essential oils are either massaged into the skin or can be inhaled. In some cases the phyto materials are heated in order to released the essential oils therefrom. By heating these phyto materials at predetermined temperatures, essential oils and extracts are boiled off, depending upon the temperature at which these phyto materials are heated, an aroma or vapor is given off, which is then inhaled by a user for its therapeutic benefits. Devices that provide such operation are generally known as vaporizers. Different phyto materials release vapors at different temperatures. Some release vapors at 120 degrees Celsius, whereas others at 220 degrees Celsius. Ideally the predetermined temperature is less than a combustion temperature of the phyto material.

More recently, there has been a trend in extracting of essential oils from the ground leaf materials and using these extracts inside vaporization devices. These extracts are also usually in a consistency honey or wax and are very messy to work with. They typically leave behind a residue, are sticky to work with and generally lack in cleanliness when they are used.

It is therefore an object of the invention to provide an aromatherapy vaporization device that overcomes the aforementioned deficiencies.

### SUMMARY

In accordance with the embodiments of the invention there is provided. An aromatherapy vaporization device comprising: a housing comprising an inhalation aperture in fluid communication with a vaporization chamber for receiving a aromatherapy material container comprising at least one of a phyto material and phyto material extract; a spike comprising a heating element thermally coupled with the spike for heating of the spike to a predetermined temperature, the spike for operating in a first mode of operation and a second mode of operation wherein the first mode of operation is for being in a cocked stated and the second mode of operation for being in a fired state; a battery; a control circuit electrically coupled with the heating element and the battery, the control circuit for receiving electrical energy from the battery and for providing of electrical current to the heating element in order to heat the heating element to the predetermined temperature in the first mode of operation of operation; a latching mechanism for maintaining the spike in the first mode of operation of operation; a trigger coupled with the housing and the latching mechanism for being activated for releasing the spike from the first mode of operation to the second mode of operation of operation, wherein, in use, upon actuating of the trigger, the latching mechanism releases the spike from the first mode of operation to the second mode of operation the spike at the predetermined temperature contacts the phyto material contained within the aromatherapy material container and transfers a portion of thermal energy from the spike to the phyto material and causes phyto material vapor to be emitted into the vaporization chamber.

In accordance with the embodiments of the invention there is provided a method of aromatherapy comprising: providing a housing comprising a vaporization chamber; providing an inhalation aperture in fluid communication with the vaporization chamber; inserting an aromatherapy material container comprising phyto material container therein into the vaporization chamber; heating of a spike to a predetermined temperature; inserting the spike at the predetermined temperature into the aromatherapy material container; transferring a portion of thermal energy from the spike to the phyto material to create a vapor derived from the phyto material; releasing of the vapor derived from the phyto material into the vaporization chamber; and, inhaling of the vapor derived from the phyto material from the inhalation aperture.

In accordance with the embodiments of the invention there is provided a An aromatherapy vaporization device comprising: a housing comprising an inhalation aperture in fluid communication with a vaporization chamber for receiving a aromatherapy material container comprising a phyto material; a spike having a low thermal inertia thermally coupled with a heating element for heating of the spike to a predetermined temperature within a predetermined amount of time, wherein the spike operates in a first mode of operation and a second mode of operation; a battery; and, a control circuit electrically coupled with the heating element and the battery, the control circuit for receiving electrical energy from the battery and for providing of electrical current to the heating element in order to heat the spike to the predetermined temperature, wherein, in use, the spike heated to the predetermined temperature is inserted into the aromatherapy material container in the second mode of operation and contacts the phyto material and results in a vapor derived from the phyto material to be emitted into the vaporization chamber for inhalation from the inhalation aperture.

### BRIEF DESCRIPTION OF THE DRAWINGS

FIG. 1A illustrates a side view of an inside detail of a preferred embodiment of the invention, a heated spike vapor delivery system (NSVD) in a first mode of operation;

FIG. 1B illustrates a side view of an inside detail of a preferred embodiment of the invention, a heated spike vapor delivery system in a second mode of operation;

FIG. 1C illustrates the vaporization chamber including an aromatherapy material container that comprises phyto material 107a contained therein;

FIG. 1D illustrates a heated spike vapor delivery system with the spike extended into the aromatherapy material container;

FIG. 2A illustrates a cutaway view of an aromatherapy vaporization device (AVD) that includes the heated spike vapor delivery system in a first mode of operation;

FIG. 2B illustrate a cutaway view of an aromatherapy vaporization device (AVD) that includes the heated spike vapor delivery system in a second mode of operation;

FIG. 3A illustrates an aromatherapy material container having the phyto material therein as well as having a covering;

FIG. 3B illustrates an aromatherapy vaporization device (AVD) in accordance a second embodiment of the invention, where a vaporization chamber is in line with an inhalation aperture and a spike; and,

FIG. 3C illustrates an aromatherapy vaporization device (AVD) in accordance a second embodiment of the invention and in a fired mode;

FIG. 4A illustrates an aromatherapy vaporization device (AVD) in accordance with a third embodiment of the invention, where the aromatherapy material container is replaced with a plurality of aromatherapy material containers; and,

FIG. 4B illustrates an aromatherapy vaporization device (AVD) in accordance with a third embodiment of the invention where one of the plurality of the aromatherapy material containers comprising vaporized phyto material contained therein is ejected out of the vaporization chamber.

### DETAILED DESCRIPTION OF EMBODIMENTS OF THE INVENTION

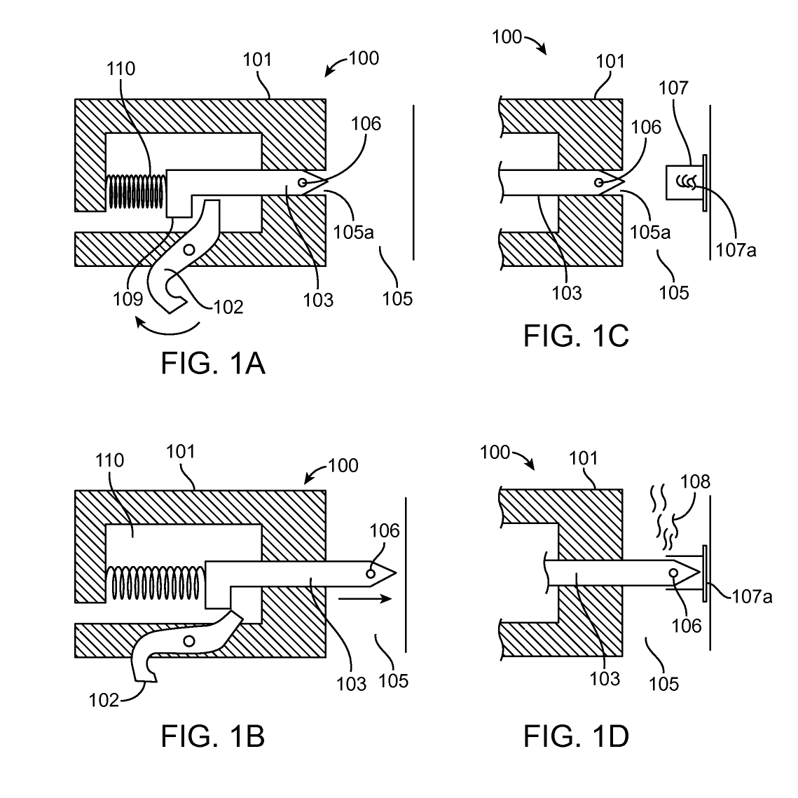

FIG. 1a illustrates a side view of an inside detail of a preferred embodiment of the invention, a heated spike vapor delivery system (HSVD) 100. The HSVD 100 is comprised of a housing 101 comprising therein a vaporization chamber 105 and having a spike 103 and a trigger 102, whereby the trigger 102 is coupled with the spike 103 using a latching mechanism 109 for having a first mode of operation, or a cocked mode of operation (FIG. 1A) and a second mode of operation, or a fired mode of operation (FIG. 1B), wherein in the cocked mode of operation the spring 110 is compressed and in the fired mode of operation the spring 110 is extended. Thermally coupled with the spike 103 is a heating element 106, which is used for heating of the spike 106 to a predetermined temperature. When the spike 110 transitions first mode of operation to the second mode of operation, potential energy is released in the spring 110. Preferably the predetermined temperature is less than a combustion temperature of the phyto material 107a. Typically phyto material combust at temperatures greater than 451 degrees Fahrenheit.

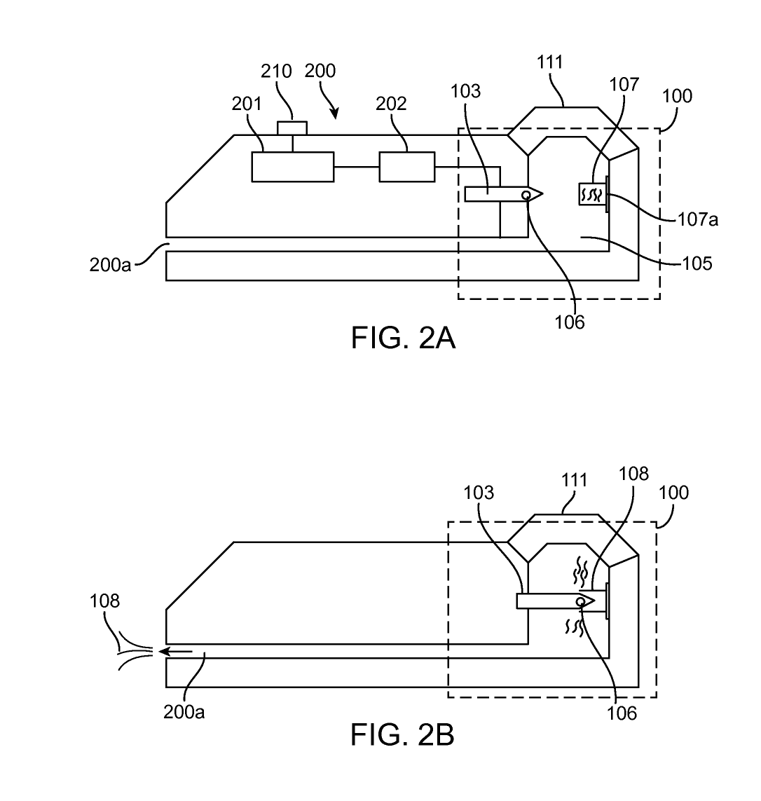

The heating element 106 is thermally coupled with the spike 103 and receives electrical current from a control circuit 202 (FIG. 2A), which is coupled with a battery 201. The control circuit 202 controls heating in order to heat up the spike 103 to the predetermined temperature in a predetermined amount of time. Preferably the spike 103 is fabricated from a metal material, however using a ceramic or glass material is also envisaged. Preferably the predetermined amount of time is less than 20 seconds.

Referring to FIGS. 1A and 1B, the vaporization chamber 105 comprises an aperture 105a through which the spike 103 one of protrudes (FIG. 1B) and other than protrudes (FIG. 1A) between the cocked mode of operation (FIG. 1A) and the fired mode of operation (FIG. 1B) of operation, respectively. In the fired mode of operation (FIG. 1B) the spike 103 is more than partially extended into the vaporization chamber 105.

FIG. 1C illustrates the vaporization chamber 105 including an aromatherapy material container 107 that comprises phyto material 107a contained therein. The phyto material 107a is in the form of either ground plant leaves or extracts of essential oils from plant leaves, or a combination of the two.

FIG. 1D illustrates the HSVD 100 with the spike 103 extended into the aromatherapy material container 107 wherein as the spike 103 at the predetermined temperature comes into contact with the phyto material 107a within the aromatherapy material container 107 and a portion of thermal energy from the spike 103 is transferred to the phyto material 107a.

This portion of thermal energy transferred as well as the predetermined temperature of the spike 103 preferably results in the phyto material 107a being heated to create a vapor derived from the phyto material 108. Preferably the aromatherapy material container 107 comprises a first diameter proximate the spike 103 and the spike 103 comprises a second diameter, wherein the second diameter is less than the first diameter and thus allows for the spike 103 to partially enter within the aromatherapy material container 107.

Whether the phyto material 107a is vaporized or combusted depends on the predetermined temperature of the spike 103. Preferably the predetermined temperature of the spike is around 190 degrees Celsius to about 220 degrees Celsius, or around 350 degrees Fahrenheit to 425 degrees Fahrenheit. Once the spike 103 contacts the phyto material 107a and the thermal energy from the spike 103 is partially transferred to the phyto material 107a it preferably releases a vapor derived from the phyto material 108.

FIGS. 2A and 2B illustrate a cutaway view of an aromatherapy vaporization device (AVD) 200 that includes the HSVD 100. As part of the AVD 200 there is included a control circuit 202 coupled with the battery 201 and the heating element 106. The vaporization chamber 105 is in fluid communication with an inhalation aperture 200a from which the vapor derived from the phyto material 108 is provided when the AVD 200 is in the fired mode of operation and the aromatherapy material 107a has come in contact with the spike 106, such as that shown in FIG. 2B. The inhalation aperture 200a is in fluid communication with the vaporization chamber 105.

Preferably the AVD includes a removable lid 111 for facilitating access to the vaporization chamber 105 for cleaning thereof. An electrical switch 210 is electrically coupled with the control circuit for controlling of electrical power being provided from the battery 201.

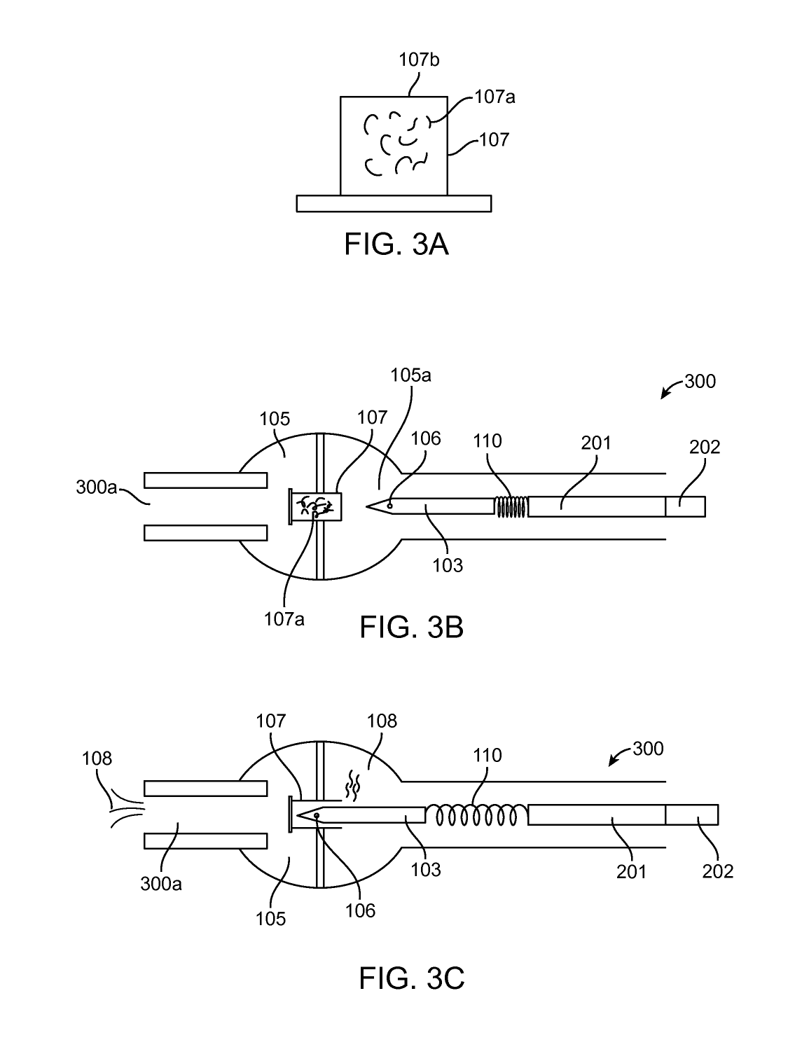

FIG. 3A illustrates the aromatherapy material container 107 having the phyto material 107a therein as well as having a covering 107b. The covering 107n facilitates loading of the aromatherapy materials into the aromatherapy material container 107 first, for example in a packaging facility, and then the covering 107b is applied to preferably form an air tight seal so that aromatic hydrocarbons do not escape from the phyto material 107a packaged in the aromatherapy material container 107. For example the covering 107b is made from a thin aluminum foil that is crimped around an outside surface of the aromatherapy material container 107.

When the AVD 100 is used with the covering 107b, the spike 103 is first heated to the predetermined temperature and loaded into the vaporization chamber in the cocked mode of operation (FIG. 2A) and then in the fired mode of operation (FIG. 2B) the spike 103 punctures the covering 107b and has a tip thereof forced into the aromatherapy material container 107 and the aromatherapy material 107a is vaporized or combusted and releases the vapor derived from the phyto material 108 into the vaporization chamber 105, which is then available for being inhaled by a user from the inhalation aperture 200a that is in fluid communication with the vaporization chamber 105 (FIG. 2B).

FIG. 3B illustrates an AVD 300 in accordance a second embodiment of the invention, where the vaporization chamber 105 is in line with an inhalation aperture 300a and the spike 103. FIG. 3b illustrates the AVD 300 in the cocked mode of operation and FIG. 3c illustrates the AVD in the fired mode. In this embodiment, the AVD 300 preferably has a vaporization chamber that is manufactured from a transparent material, such as glass or transparent plastic.

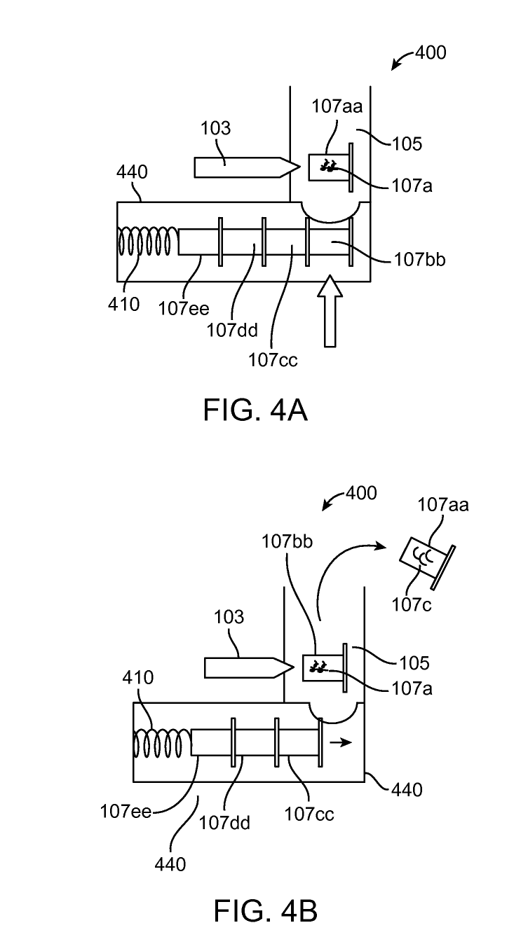

FIG. 4A illustrates the AVD 400 in accordance with a third embodiment of the invention, where the aromatherapy material container 107 is replaced with a plurality of aromatherapy material containers (107aa through 107ee), where these aromatherapy material container are loaded into a magazine 440 and are inserted into the vaporization chamber 105 during the cocked mode of operation and then vaporized during the fired mode of operation of operation.

After completing of the fired mode of operation of operation, when returning to the cocked mode of operation of operation, one of the plurality of the aromatherapy material containers 107aa comprising vaporized vaporized phyto material 107c contained therein is ejected out of the vaporization chamber 105 and another aromatherapy material container 107bb is inserted in to the vaporization chamber and is ready for being vaporized when the AVD 400 returns to the fired mode of operation, as is shown in FIG. 4B, when the trigger (102 FIG. 1a) is actuated and the latching mechanism allows for the spike 103 to pierce the aromatherapy material container covering 107b and then to come into contact with the phyto material 107a to release vapor therefrom.

Preferably the spike comprises a low thermal inertia material construction and heats up to the predetermined temperature in less than 20 seconds. An example of such a material is a hollow copper tube that is closed on one end and has a heating element embedded therein. The closed end of the spike is used to contact the phyto material 107a. Advantageously, having a low thermal inertia allows for the spike to attain the predetermined temperature within preferably a few seconds and this proves to have batter life savings advantages as well as allowing for fast heatup times for end user happiness.

Advantageously, the AVD 100, 300, 400 in accordance with the embodiments of the invention allows for having a quick heating time because of the spike 103 having a low thermal mass. Having a low thermal mass allows for quick heating and as a result also causes there to be less of a current draw from the battery. An amount of phyto material that is inside the aromatherapy container is preferably about 0.05 g to 0.15 g in weight.

Furthermore, advantageously because the spike 103 is not in contact with the phyto material 107a until just before the spike 103 is inserted into the aromatherapy material container 107, this allows for the phyto material 107a to maintain its flavor as well as its essential oils and other aromatic hydrocarbons until they are ready to be vaporized. In some cases with the phyto material 107a being in contact with a hot element over time may result in potency of the phyto material vapor to decrease.

Another advantage of the embodiments, having the phyto material or phyto material extracts pre packaged into the aromatherapy material container 107 at a processing factory is advantageous as it makes for less of a mess when dealing with these materials for the end user. It allows for clean handling of the aromatherapy material container 107 without handling the phyto material or phyto material extracts.

Advantageously, having the aromatherapy material container 107 allows for more accurate manner in which to a dose of phyto material 107a that is being vaporized by the user. Optionally, the aromatherapy material containers 107 are provided in volumes and the spike is optionally provided in various diameters, thus allowing for a measured dose.

Numerous other embodiments are envisaged without departing from the spirit or scope of the invention.

What I claim is:

1. An aromatherapy vaporization device comprising:
a housing comprising an inhalation aperture in fluid communication with a vaporization chamber for receiving a aromatherapy material container comprising at least one of a phyto material and phyto material extract;
a spike comprising a heating element thermally coupled with the spike for heating of the spike to a predetermined temperature, the spike for operating in a first mode of operation and a second mode of operation wherein the first mode of operation is for being in a cocked stated and the second mode of operation for being in a fired state;
a battery;
a control circuit electrically coupled with the heating element and the battery, the control circuit for receiving electrical energy from the battery and for providing of electrical current to the heating element in order to heat the heating element to the predetermined temperature in the first mode of operation of operation;
a latching mechanism for maintaining the spike in the first mode of operation of operation;
a trigger coupled with the housing and the latching mechanism for being activated for releasing the spike from the first mode of operation to the second mode of operation of operation,
wherein, in use, upon actuating of the trigger, the latching mechanism releases the spike from the first mode of operation to the second mode of operation the spike at the predetermined temperature contacts the phyto material contained within the aromatherapy material container and transfers a portion of thermal energy from the spike to the phyto material and causes phyto material vapor to be emitted into the vaporization chamber.

2. An aromatherapy vaporization device according to claim 1 wherein the housing comprises a removable lid for facilitating access to the vaporization chamber for inserting and removing of the removing of the aromatherapy material container therefrom.

3. An aromatherapy vaporization device according to claim 1 wherein the aromatherapy material container comprises a covering for encapsulating the phyto material within the aromatherapy material container and when upon actuating of the trigger, the latching mechanism releases the spike from the first mode of operation to the second mode of operation and the spike at the predetermined temperature pierces the covering and contacts the phyto material contained within the aromatherapy material container and causes phyto material vapor to be emitted into the vaporization chamber.

4. An aromatherapy vaporization device according to claim 4 comprising a spring, wherein the spring is coupled with the spike and the housing, wherein the spring releases potential energy when the spike transitions between the first mode of operation to the second mode of operation.

5. An aromatherapy vaporization device according to claim 1 wherein the vaporization chamber comprising an aperture through which the spike protrudes in the second mode of operation and other than protrudes in the first mode of operation.

6. An aromatherapy vaporization device according to claim 1 wherein the spike comprises a low thermal inertia material construction and heats up to the predetermined temperature in less than 20 seconds.

7. An aromatherapy vaporization device according to claim 6 wherein the aromatherapy material container comprises a first diameter proximate the spike and the spike comprises a second diameter, wherein the second diameter is less than the first diameter.

8. A method of aromatherapy comprising:
providing a housing comprising a vaporization chamber;
providing an inhalation aperture in fluid communication with the vaporization chamber;
inserting an aromatherapy material container comprising phyto material container therein into the vaporization chamber;
heating of a spike to a predetermined temperature;
inserting the spike at the predetermined temperature into the aromatherapy material container;
transferring a portion of thermal energy from the spike to the phyto material to create a vapor derived from the phyto material;
releasing of the vapor derived from the phyto material into the vaporization chamber; and,
inhaling of the vapor derived from the phyto material from the inhalation aperture.

9. A method of aromatherapy according to claim 8 wherein the predetermined temperature is less than a combustion temperature of the phyto material.

10. A method of aromatherapy according to claim 9 wherein the predetermined temperature is between 200 degrees Celcius and 230 degrees Celcius.

11. A method of aromatherapy according to claim 8 wherein transferring a portion of thermal energy from the spike to the phyto material comprises providing a trigger for controlling of the spike inserting into the aromatherapy material container.

12. A method of aromatherapy according to claim 8 comprising ejecting the aromatherapy material container comprising vaporized phyto material contained therein out of the vaporization chamber.

13. An aromatherapy vaporization device comprising:
a housing comprising an inhalation aperture in fluid communication with a vaporization chamber for receiving a aromatherapy material container comprising a phyto material;
a spike having a low thermal inertia thermally coupled with a heating element for heating of the spike to a predetermined temperature within a predetermined amount of time, wherein the spike operates in a first mode of operation and a second mode of operation;
a battery; and,
a control circuit electrically coupled with the heating element and the battery, the control circuit for receiving electrical energy from the battery and for providing of electrical current to the heating element in order to heat the spike to the predetermined temperature,
wherein, in use, the spike heated to the predetermined temperature is inserted into the aromatherapy material container in the second mode of operation and contacts the phyto material and results in a vapor derived from the phyto material to be emitted into the vaporization chamber for inhalation from the inhalation aperture.

14. An aromatherapy vaporization device according to claim 13 comprising a latching mechanism for maintaining the spike in the first mode of operation of operation and for releasing of the spike into the second mode of operation.

15. An aromatherapy vaporization device according to claim 14 comprising a spring coupled with the spike for facilitating the insertion of the spike into the aromatherapy material container.
